(19) United States
(12) Patent Application Publication
(10) Pub. No.: US 2009/0118774 A1
(43) Pub. Date: May 7, 2009

(54) Sternal Closure Device with Ratchet Closure Mechanism

(75) Inventor: Archibald S. Miller III, Tulsa, OK (US)

Correspondence Address: CROWE AND DUNLEVY, P.C., 20 NORTH BROADWAY, SUITE 1800, OKLAHOMA CITY, OK, 73102-8273, US

(73) Assignee: MAVREK MEDICAL, LLC., TULSA, OK (US)

(21) Appl. No.: 11/884,171

(22) PCT Filed: Feb. 9, 2006
(86) PCT No.: PCT/US06/04819

§ 371 Date: Apr. 15, 2008

Related U.S. Application Data
(60) Provisional application No. 60/651,580, filed on Feb. 9, 2005.

(51) Int. Cl. A61B 17/84

(52) U.S. Cl. 606/324

(57) ABSTRACT

A sternal closure device includes an insertion member, a receiving member and a lock assembly. The insertion member includes a first series of laterally disposed teeth-like projections along a first side of the insertion member and the lock assembly includes a spring that is configured to engage the first series of laterally disposed teeth-like projections of the insertion member. The lock assembly is removably secured to a first side of the receiving member.

### RELATED APPLICATIONS

The present application claims the benefit of U.S. Provisional Patent Application No. 60/651,580, filed Feb. 9, 2005, entitled “Single-Leg Sternal Closure Device,” the disclosure of which is herein incorporated by reference.

### FIELD OF INVENTION

The present invention relates generally to surgical positioning devices and more particularly to a surgical device for capturing, positioning and aligning portions of a severed human sternum.

### BACKGROUND OF INVENTION

Wire closures are widely represented in the contemporary art as a recognized means for closing the sternum following a mid-line sternotomy. Nonetheless, wire-based closure devices suffer from several deficiencies. First, the average implementation/deployment time 15-20 minutes per mechanism. Such an excessive time requirement translates, of course, into increased operating room costs, increased doctor, staff and insurance costs, and increased doctor fatigue. Second, a variety of disruptive forces exasperate the very intention of wire-based systems. Forces associated with sternotomies include the rectus abdominus muscle exerting an uneven pull, a lateral pull by pectoralis muscles, forces generated by valsalva action and anterior/posterior displacement of hemi-sternum via respiratory muscle action.

In view of the deficiencies of wire based clamping surgical devices, and further in view of the life threatening conditions associated with failure of such devices, newer technology has been recognized as superior to the wire devices. Such devices are generically referred to as clamp closures. Clamp closures typically include two primary elements that can be removably associated with one another. As in U.S. Pat. No. 6,051,007 issued Apr. 18, 2000 to Hogendijk et al., (“Hogendijk '007”), several prior art clamp closures include two clamp elements that each include a “leg” portion configured to grasp an opposing side of a vertically severed sternum. When approximated, the two clamp elements draw the severed sternum into closed position. A locking feature is used to hold the clamp closures in the approximated position.

Although generally effective, prior art clamp closures suffer several deficiencies. For example, many locking mechanisms used in prior art closures fail under increased stress loads. Other locking mechanisms are difficult to manipulate or impossible to quickly remove under emergency conditions. It is to these and other deficiencies in the prior art that the present invention is directed.

### SUMMARY OF THE INVENTION

In a preferred embodiment, the present invention includes a sternal closure device having an insertion member, a receiving member and a lock assembly. The insertion member includes a first series of laterally disposed teeth-like projections along a first side of the insertion member and the lock assembly includes a spring that is configured to engage the first series of laterally disposed teeth-like projections of the insertion member. The lock assembly is removably secured to a first side of the receiving member.

### BRIEF DESCRIPTION OF DRAWINGS

FIG. 1 is a front perspective view of a sternal closure device constructed in accordance with a preferred embodiment of the present invention in an “open” position.

FIG. 2 is a rear perspective view of the sternal closure device of FIG. 1 in a “closed” position.

FIG. 3 is a rear perspective view of the insertion portion of the sternal closure device of FIG. 1.

FIG. 4 is a front perspective view of the receiving portion of the sternal closure device of FIG. 1.

FIG. 5 is a rear perspective view of the receiving portion of the sternal closure device of FIG. 1 with the lock assembly removed.

FIG. 6 is a front perspective view of the sternal closure device of FIG. 1 in a closed position with the lock assembly removed.

FIG. 7 is a close-up top perspective view of the insertion portion and lock assembly of the sternal closure device of FIG. 1.

FIG. 8 is a close-up top perspective view of the sternal closure device of claim 1 in a “closed” position showing the engagement between the lock assembly and the insertion portion.

FIG. 9 is a front perspective view of a sternal closure device constructed in accordance with an alternate embodiment of the present invention.

### DETAILED DESCRIPTION OF PREFERRED EMBODIMENTS

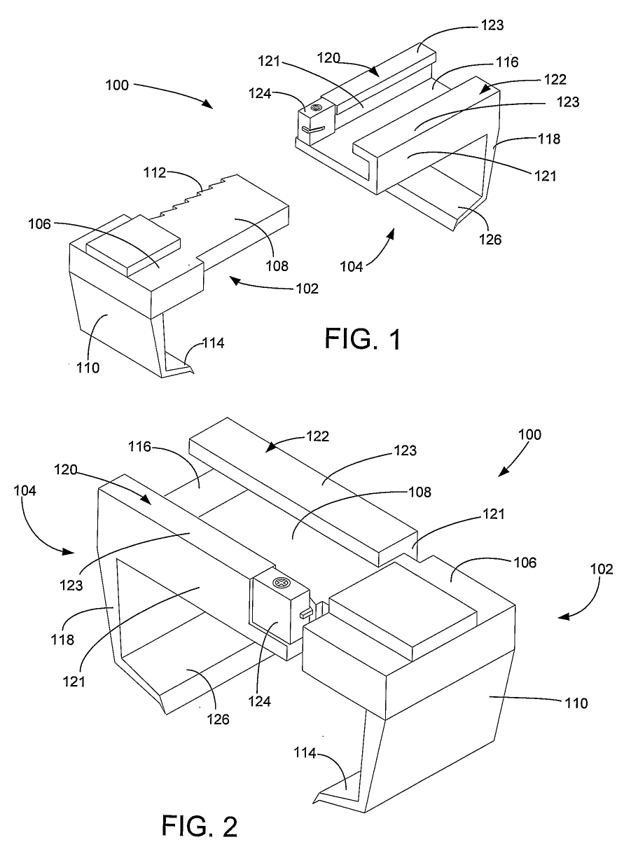

Referring to FIG. 1, shown therein is a front perspective view of a sternal closure device 100 constructed in accordance with a presently preferred embodiment in an “open” position. FIG. 2 provides a rear perspective view of the sternal closure device 100 in a “closed” position. In the preferred embodiment, the sternal closure device 100 includes an insertion member 102 and a receiving member 104. As disclosed below, the preferred embodiment of the sternal closure device 100 includes a number of components that cooperatively perform the intended function of positioning, aligning and securing a severed sternum. It will be understood, however, that the disclosure herein is of a single preferred embodiment and that alternate embodiments with additional features are also encompassed within the scope of the present invention.

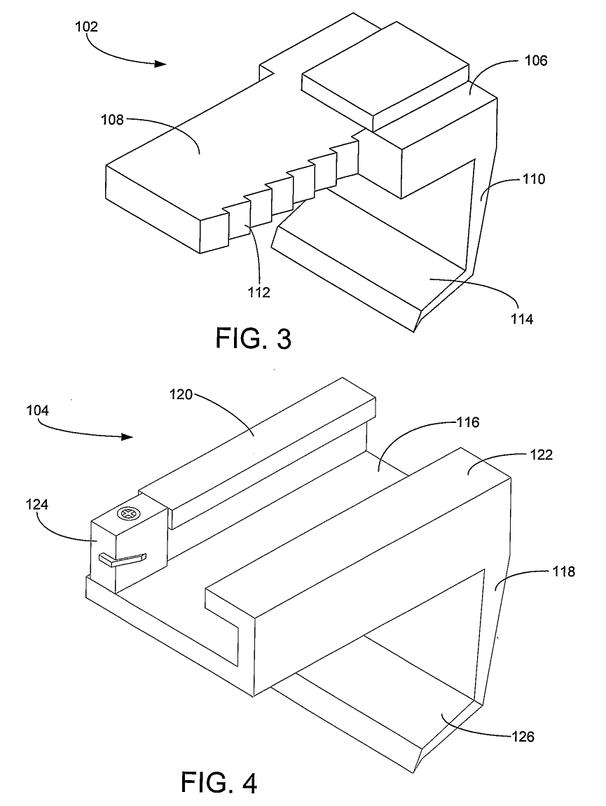

Continuing with FIG. 1, and also referring to FIG. 3, the insertion member 102 includes a base 106, a key 108 and a leg portion 110. In the preferred embodiment, the base 106, key 108 and leg portion 110 are formed as an integral piece from a durable, rigid material. In a particularly preferred embodiment, the insertion member 102 is constructed from a suitable surgical-grade metal alloy, ceramic or hardened plastic.

In the preferred embodiment shown in FIG. 3, the key 108 includes a first series of teeth-like projections 112 along a first side of the key 108. The size, shape and number of teeth-like projections 112 may vary with application. It is preferred, however, that the key 108 include 10 to 50 teeth-like projections. Increasing the number of teeth-like projections 112 increases the number of positions in which the insertion member 102 can be secured within the receiving member 104. The leg portion 110 is preferably configured as a substantially crescent-shaped member that includes a downwardly disposed foot portion 114. In the preferred embodiment, the crescent shape of the leg portion 110 is shown as a series of straight sections connected at acute angles.

Referring now also to FIG. 4, the receiving member 104 preferably includes a receiver base 116, a leg portion 118, a first guide wall 120, a second guide wall 122 and a lock assembly 124. In the preferred embodiment, the receiver base 116, leg portion 118, first guide wall 120 and second guide wall 122 are formed as an integral piece from a durable, rigid material. In a particularly preferred embodiment, these elements of the receiving member 104 are constructed from a suitable surgical-grade metal alloy, ceramic or hardened plastic.

The leg portion 118 is preferably configured as a substantially crescent-shaped member that includes a downwardly disposed foot portion 126. In the preferred embodiment, the crescent shape of the leg portion 118 is shown as a series of straight sections connected at acute angles that geometrically mirrors the leg portion 110 of the insertion member 102.

The first and second guide walls 120, 122 are preferably configured to receive the key 108 of the insertion member 102. The first and second guide walls 120, 122 preferably include vertical and horizontal portions 121, 123, respectively, that are sized and configured to restrict the movement of the key 108 to a single axis when engaged, as shown in FIG. 2. In this way, the vertical and horizontal portions 121, 123 form channels configured to control the movement of the key 108 within the receiving member 104.

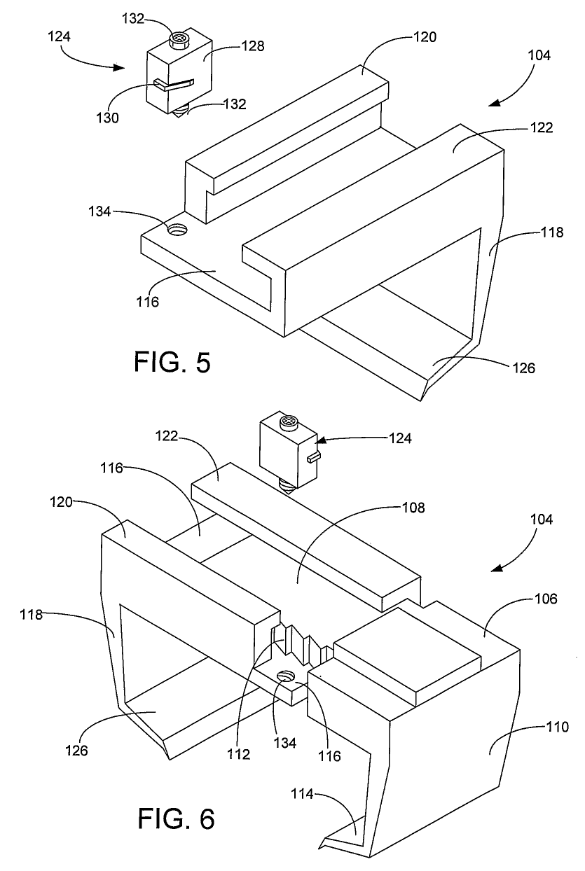

Turning to FIG. 5, shown therein is the receiving member 104 with the lock assembly 124 in a removed position. In the presently preferred embodiment, the lock assembly 124 includes a block 128, a spring 130 and a fastener 132. The block 128 is preferably sized and configured to be positioned adjacent the first guide wall 120. The fastener 132 is preferably a screw-type fastener that secures the lock assembly 124 to the base 116 of the receiving member 104. In a particularly preferred embodiment, the base 116 includes a threaded bore 134 that is configured to receive the fastener 132. Although a screw-type fastener is presently preferred, it will be understood that alternative fasteners could also be used to secure the lock assembly 124 to the receiving member 104. It is preferred that the top of the screw-type fastener 132 is substantially flush with the top surface of the block 128 when the fastener 132 is fully engaged. It is also preferred that, when installed, a vertical side of the lock assembly 124 abut the first guide wall 120. In this way, the lock assembly 124 is prevented from rotating on the receiver base 116 about the threaded bore 134.

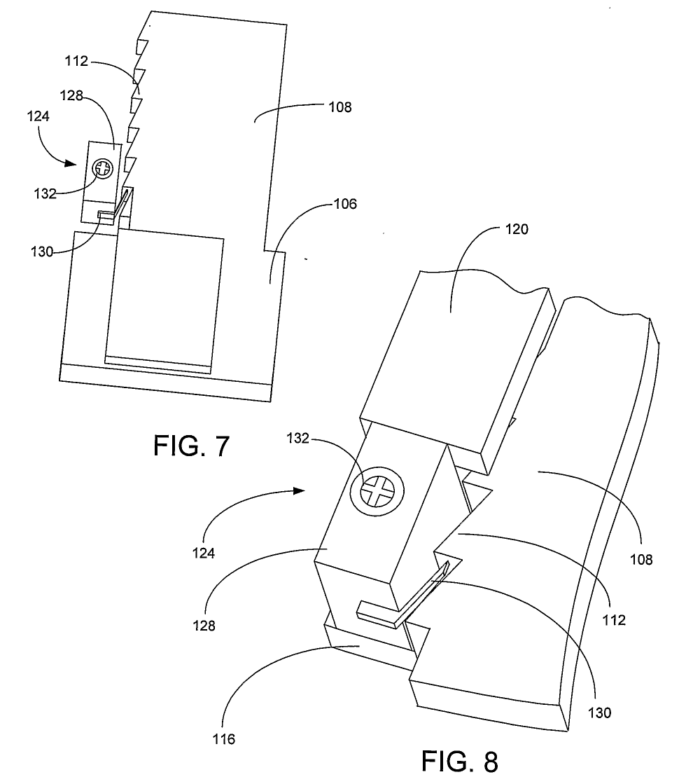

Referring now to FIGS. 7 and 8, the spring 130 is preferably constructed from a resilient material, such as stainless steel. In a particularly preferred embodiment, the spring 130 and block 128 are constructed as a unitary piece. The spring 130 is preferably configured to engage the teeth-like projections 112 of the key 108. The spring 130 flexes to allow the individual teeth-like projections 112 to pass as the insertion member 102 is introduced into the receiving member 104. As each teeth-like structure 112 passes beyond the extent of the spring 130, the spring 130 returns to its relaxed position. The geometry of the teeth-like projections 112 and the spring 130 cooperatively prohibit the removal of the insertion member 102 from the receiving member 104. In this arrangement, the spring 130 and teeth-like projections 112 behave similarly to a common ratchet-and-pawl mechanism.

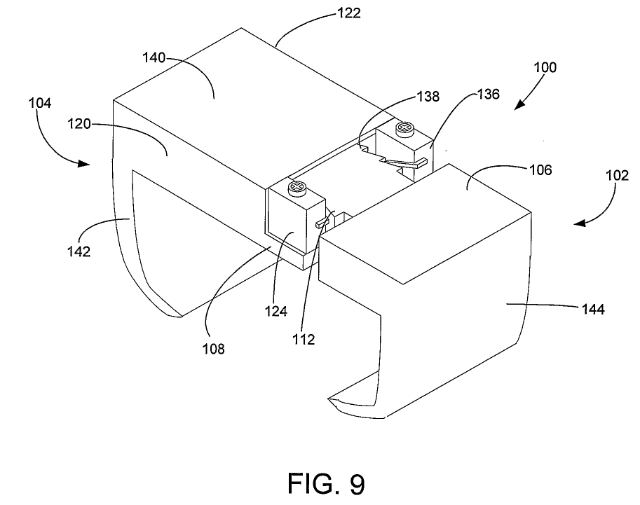

In an alternate preferred embodiment shown in FIG. 9, the sternal closure device 100 includes a second lock assembly 136 on a second side of the receiving member 104 and the insertion member 102 includes a second series of teeth-like projections 138 on the second side of the key 108. Unlike the discrete horizontal portions 123 of the first and second guide walls 120, 122, a single unitary cover 140 joins the vertical portions 121. The cover 140 spans the width of the receiving member 106, thereby forming a substantially closed receiving chamber.

The crescent-shape of the leg portions 142, 144 of the alternate embodiment of FIG. 9 are configured as a curvaceous or arcuate members. It will be appreciated that discrete components from the separate embodiments disclosed herein can be successfully combined to yield a sternal closure device 100 designed for specific applications. For example, it may be desirable to employ the curvaceous leg portions 142, 144 in combination with a single lock assembly 124 and single series of teeth-like projections 112.

In use, the insertion member 102 and receiving member 104 contact opposing portions of the severed sternum. As the sternum is approximated, the insertion member 102 is introduced into the receiving member 104. As the key 108 passes by the lock assembly 124, the teeth-like projections 112 interact with the spring 130 according to the ratchet-and-pawl mechanism. The forward progress of the insertion member 102 into the receiving member 104 is primarily opposed by the closure of the opposing sternal portions. The withdrawal of the insertion member 102 from the receiving member 104 is prevented by the blocking presence of the spring 130 against the teeth-like projections 112. To release the insertion member 102, the lock assembly 124 must be removed from the receiving member 104. In the presently preferred embodiment, the lock assembly 124 is removed by releasing the fastener 132 with a screwdriver or similar tool.

It is clear that the present invention is well adapted to carry out its objectives and attain the ends and advantages mentioned above as well as those inherent therein. While presently preferred embodiments of the invention have been described in varying detail for purposes of disclosure, it will be understood that numerous changes may be made which will readily suggest themselves to those skilled in the art and which are encompassed within the spirit of the invention disclosed herein and in the associated drawings and appended claims.

1. A sternal closure device comprising:
an insertion member, wherein the insertion member includes a first series of laterally disposed teeth-like projections along a first side of the insertion member;
a receiving member; and
a lock assembly removably secured to a first side of the receiving member, wherein the lock assembly includes a block and a spring configured to engage the first series of laterally disposed teeth-like projections of the insertion member, and wherein the spring extends from the block.

2. The sternal closure device of claim 1, wherein the insertion member includes a second series of laterally disposed teeth-like projections on a second side of the insertion member.

3. The sternal closure device of claim 2, further comprising a second lock assembly removably secured to the second side of the receiving member, wherein the second lock assembly includes a second block and a second spring configured to engage the second series of laterally disposed teeth-like projections of the insertion member, and wherein the second spring extends from the second block.

4. The sternal closure device of claim 3, wherein the first and second lock assemblies are removably secured to the receiving member with fasteners.

5. The sternal closure device of claim 4, wherein the fasteners are screw-type fasteners and the receiving member includes threaded bores configured to receive the screw-type fasteners.

6. The sternal closure device of claim 1, wherein the insertion member includes a leg portion and the receiving member includes a leg portion.

7. The sternal closure device of claim 1, wherein the receiving member further comprises a base, a first guide wall proximate the first side of the receiving member and a second guide wall proximate the second side of the receiving member.

8. The sternal closure device of claim 7, wherein the first and second guide walls each include a vertical portion attached to the base and a horizontal portion attached to the vertical portion.

9. The sternal closure device of claim 8, wherein the horizontal portions of the first and second guide walls form a single horizontal portion that spans the distance between first and second sides of the receiving member.
